SEQUENCE LISTING (45 sequences)

SEQ ID NO: 1 - protein, 39 aa, Heloderma suspectum
  MOD_RES (39)..(39): Ser is amidated
HGEGTFTSDLSKQMEEEAVRLFIEWLKNGGPSSGAPPPS

SEQ ID NO: 2 - protein, 39 aa, Heloderma horridum
  MOD_RES (39)..(39): Ser is amidated
HSDGTFTSDLSKQMEEEAVRLFIEWLKNGGPSSGAPPPS

SEQ ID NO: 3 - protein, 39 aa, Artificial Sequence
  synthetic peptide construct
HGEGTFTSDLSKQLEEEAVRLFIEWLKNGGPSSGAPPPS

SEQ ID NO: 4 - protein, 39 aa, Artificial Sequence
  synthetic peptide construct
HGEGTFTSDLSKQLEEEAVRLFIEFLKNGGPSSGAPPPS

SEQ ID NO: 5 - protein, 39 aa, Artificial Sequence
  synthetic peptide construct
HGEGTFTSDLSKQLEEEAARLFIEFLKNGGPSSGAPPPS

SEQ ID NO: 6 - protein, 30 aa, Artificial Sequence
  synthetic peptide construct
HGEGTFTSDLSKQMEEEAVRLFIEWLKNGG

SEQ ID NO: 7 - protein, 30 aa, Artificial Sequence
  synthetic peptide construct
HGEGTFTSDLSKQLEEEAVRLFIEWLKNGG

SEQ ID NO: 8 - protein, 30 aa, Artificial Sequence
  synthetic peptide construct
HGEGTFTSDLSKQLEEEAVRLFIEFLKNGG

SEQ ID NO: 9 - protein, 30 aa, Artificial Sequence
  synthetic peptide construct
HGEGTFTSDLSKQLEEEAARLFIEFLKNGG

SEQ ID NO: 10 - protein, 28 aa, Artificial Sequence
  synthetic peptide construct
HGEGTFTSDLSKQMEEEAVRLFIEWLKN

SEQ ID NO: 11 - protein, 28 aa, Artificial Sequence
  synthetic peptide construct
HGEGTFTSDLSKQLEEEAVRLFIEWLKN

SEQ ID NO: 12 - protein, 28 aa, Artificial Sequence
  synthetic peptide construct
HGEGTFTSDLSKQLEEEAVRLFIEFLKN

SEQ ID NO: 13 - protein, 28 aa, Artificial Sequence
  synthetic peptide construct
HGEGTFTSDLSKQLEEEAARLFIEFLKN

SEQ ID NO: 14 - protein, 39 aa, Artificial Sequence
  synthetic peptide construct
HGEGTFTSDLSKQLEEKAAKEFIEFLKQGGPSSGAPPPS

SEQ ID NO: 15 - protein, 39 aa, Artificial Sequence
  synthetic peptide construct
HGEGTFTSDLSKQLEEKAAKEFIEWLKQGGPSSGAPPPS

SEQ ID NO: 16 - protein, 39 aa, Artificial Sequence
  synthetic peptide construct
HGEGTFTSDLSKQXEEEAVRLFIEWLKQGGPSSGAPPPS

SEQ ID NO: 17 - protein, 39 aa, Artificial Sequence
  synthetic peptide construct
HGEGTFTSDLSKQLEEEAVRLFIEWLKQGGPSSXAPPPS

SEQ ID NO: 18 - protein, 37 aa, Artificial Sequence
  synthetic peptide construct
HGEFTFTSDLSKQLEEEAVRLFIEWLKQGGPSKEIIS

SEQ ID NO: 19 - protein, 39 aa, Artificial Sequence
  synthetic peptide construct
HGEFTFTSDLSKQLEEKAAKEFIEWLKQGGPSSGAPPPS

SEQ ID NO: 20 - protein, 39 aa, Artificial Sequence
  synthetic peptide construct
HGEGTFTSDLVKILEAEAVRKFIEFLKNGGPSSGAPPPS

SEQ ID NO: 21 - protein, 40 aa, Artificial Sequence
  synthetic peptide construct
HGEGTFTSDLSKQMEEEAVRLFIEWLKNGGPSSGAPPPSK

SEQ ID NO: 22 - protein, 31 aa, Homo sapiens
  MOD_RES (31)..(31): Gly is optionally amidated
HAEGTFTSDVSSYLEGQAAKEFIAWLVKGRG

SEQ ID NO: 23 - protein, 30 aa, Homo sapiens
  MOD_RES (30)..(30): Arg is optionally amidated
HAEGTFTSDVSSYLEGQAAKEFIAWLVKGR

SEQ ID NO: 24 - protein, 30 aa, Artificial Sequence
  synthetic peptide construct
HXEGTFTSDVSSYLEGQAAREFIAFLVRXR

SEQ ID NO: 25 - protein, 31 aa, Artificial Sequence
  synthetic peptide construct
HXEGTFTSDVSSYLEXXAAKEFIXWLXXGXX

SEQ ID NO: 26 - protein, 31 aa, Artificial Sequence
  synthetic peptide construct
HAEGTFTSDVSSYLEGQAAKEFIAWLVRGRG

SEQ ID NO: 27 - protein, 31 aa, Artificial Sequence
  synthetic peptide construct
HAEGTFTSDVSSYLEGQAAKEFIEWLVKGRG

SEQ ID NO: 28 - protein, 31 aa, Artificial Sequence
  synthetic peptide construct
HAEGTFTSDVSSYLEKQAAKEFIAWLVKGRG

SEQ ID NO: 29 - protein, 31 aa, Artificial Sequence
  synthetic peptide construct
HGEGTFTSDVSSYLEEQAAKEFIAWLVKGGG

SEQ ID NO: 30 - protein, 31 aa, Artificial Sequence
  synthetic peptide construct
HVEGTFTSDVSSYLEEQAAKEFIAWLVKGGG

SEQ ID NO: 31 - protein, 31 aa, Artificial Sequence
  synthetic peptide construct
HGEGTFTSDVSSYLEEQAAKEFIAWLKNGGG

SEQ ID NO: 32 - protein, 31 aa, Artificial Sequence
  synthetic peptide construct
HVEGTFTSDVSSYLEEQAAKEFIAWLKNGGG

SEQ ID NO: 33 - protein, 31 aa, Artificial Sequence
  synthetic peptide construct
HGEGTFTSDVSSYLEEQAAKEFIAWLVKGGP

SEQ ID NO: 34 - protein, 31 aa, Artificial Sequence
  synthetic peptide construct
HVEGTFTSDVSSYLEEQAAKEFIAWLVKGGP

SEQ ID NO: 35 - protein, 31 aa, Artificial Sequence
  synthetic peptide construct
HGEGTFTSDVSSYLEEQAAKEFIAWLKNGGP

SEQ ID NO: 36 - protein, 31 aa, Artificial Sequence
  synthetic peptide construct
HVEGTFTSDVSSYLEEQAAKEFIAWLKNGGP

SEQ ID NO: 37 - protein, 30 aa, Artificial Sequence
  synthetic peptide construct
HGEGTFTSDVSSYLEEQAAKEFIAWLVKGG

SEQ ID NO: 38 - protein, 30 aa, Artificial Sequence
  synthetic peptide construct
HVEGTFTSDVSSYLEEQAAKEFIAWLVKGG

SEQ ID NO: 39 - protein, 30 aa, Artificial Sequence
  synthetic peptide construct
HVEGTFTSDVSSYLEEQAAKEFIAWLKNGG

SEQ ID NO: 40 - protein, 30 aa, Artificial Sequence
  synthetic peptide construct
HGEGTFTSDVSSYLEEQAAKEFIAWLKNGG

SEQ ID NO: 41 - protein, 230 aa, Artificial Sequence
  synthetic peptide construct
AESKYGPPCPPCPAPXXXGGPSVFLFPPKPKDTLMISRTPEVTCVVVDVSQEDPEVQFNWYVDGVEVHNAKTKPREEQFXSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGX

SEQ ID NO: 42 - protein, 5 aa, Artificial Sequence
  synthetic peptide construct
GGGGS

SEQ ID NO: 43 - protein, 275 aa, Artificial Sequence
  synthetic peptide construct
HGEGTFTSDVSSYLEEQAAKEFIAWLVKGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 44 - protein, 40 aa, Artificial Sequence
  synthetic construct
HXEGTFTSDVSSYLEXQAAKEFIAWLXKGGPSSGAPPPCC

SEQ ID NO: 45 - protein, 40 aa, Artificial Sequence
  synthetic peptide construct
HVEGTFTSDVSSYLEEQAAKEFIAWLIKGGPSSGAPPPCC

(19) United States
(12) Patent Application Publication
(10) Pub. No.: US 2012/0231022 A1
(43) Pub. Date: Sep. 13, 2012

(54) GLP-1 RECEPTOR AGONIST COMPOUNDS FOR SLEEP ENHANCEMENT

(75) Inventors: Joseph T. Bass, Evanston, IL (US); Aaron D. Laposky, Gaithersburg, MD (US); Fred W. Turek, Chicago, IL (US)

(73) Assignee: Amylin Pharmaceuticals, Inc., San Diego, CA (US)

(21) Appl. No.: 13/318,766

(22) PCT Filed: May 27, 2010
(86) PCT No.: PCT/US10/36326

§ 371 Date: May 23, 2012

Related U.S. Application Data
(60) Provisional application No. 61/181,979, filed on May 28, 2009.

(51) Int. Cl. A61K 38/22; A61K 47/48; A61P 25/00

(52) U.S. Cl. 424/178.1; 514/9.7

(57) ABSTRACT

The disclosure provides, among other things, the use of GLP-1 receptor agonist compounds to enhance sleep, increase the duration and/or intensity of non-rapid eye movement (NREM) sleep, treat NREM sleep disorders, and to treat circadian rhythm sleep disorders. The GLP-1 receptor agonist compounds may be exendins, exendin analogs, GLP-1(7-37), GLP-1(7-37) analogs (e.g., GLP-1(7-36)-NH₂) and the like. In one embodiment, the GLP-1 receptor agonist compound is exenatide.

### RELATED APPLICATIONS

This application claims priority to U.S. Application No. 61/181,979 filed May 28, 2009.

### FIELD

The description is directed to the field of medicine, more particularly, to sleep.

### BACKGROUND

The overnight course of sleep involves the central nervous system at different levels at different times. During sleep, several states of vigilance are classified on the basis of conventional polysomnographic (PSG) measures: four non-rapid eye movement (NREM) stages and one rapid eye movement (REM) stage. The daily shifts from the wake state to NREM and REM sleep are under the control of interconnected processes, including the circadian timing of sleep onset; the homeostatic balance between wakefulness and sleep; and the ultradian interaction between NREM and REM sleep. More recently, especially to explain the clinical consequences of sleep disorders, the three processes of sleep regulation—circadian, homeostatic and ultradian—have been integrated by the definition of the arousal system. Arousals are transient episodes of cerebral activation during sleep, which involves the cortex regulated by the interplay of cortical and subcortical neurons. Arousals are considered a transient cortical activation in response to sleep disruptive events. Other studies indicate that arousals punctuate both REM and NREM sleep, even in the absence of detectable disturbing stimuli. Besides conventional arousals, other electroencephalographic (EEG) patterns, such as K-complexes and delta bursts, are associated with an analogous activation of vegetative and somatomotor functions.

Sleep quality depends on several factors, including total sleep time; sleep efficiency (e.g., total sleep time versus time spent in bed); time of sleep onset; the number and duration of night time awakenings; the number of NREM sleep cycles; and the duration of time spent in the respective sleep stages (e.g., NREM sleep stages). There exists a need in the art to identify new therapeutic agents that can be used to enhance the quality of sleep. The present disclosure is directed to these, as well as other, important ends.

### SUMMARY

Provided herein are methods to enhance sleep in patients in need thereof by administering to the patients therapeutically effective amounts of GLP-1 receptor agonist compounds to enhance sleep. In one embodiment, the method is to enhance NREM sleep.

Provided herein are methods to increase the duration of NREM sleep time in patients in need thereof by administering to the patients therapeutically effective amounts of GLP-1 receptor agonist compounds to increase the duration of NREM sleep time. In one embodiment, the methods increase the duration of uninterrupted NREM sleep time.

Provided herein are methods to increase the intensity of NREM sleep time in patients in need thereof by administering to the patients therapeutically effective amounts of GLP-1 receptor agonist compounds to increase the intensity of NREM sleep time.

Provided herein are methods to increase the duration and intensity of NREM sleep time in patients in need thereof by administering to the patients therapeutically effective amounts of GLP-1 receptor agonist compounds to increase the duration and intensity of NREM sleep time.

Provided herein are methods to treat NREM sleep disorders in patients in need thereof by administering to the patients therapeutically effective amounts of GLP-1 receptor agonist compounds to treat NREM sleep disorders. NREM sleep disorders include parasomnias. Exemplary NREM sleep disorders include sleepwalking disorder, sleep terror disorder, enuresis, sleep bruxism, restless leg syndrome, periodic limb movement disorder, and the like.

Provided herein are methods to treat or prevent circadian rhythm sleep disorders in patients in need thereof by administering to the patients therapeutically effective amounts of GLP-1 receptor agonist compounds to treat or prevent the circadian rhythm sleep disorder. One exemplary circadian rhythm sleep disorder is desynchronosis.

Provided herein are methods to enhance sleep (e.g., NREM sleep); increase the duration of NREM sleep time; increase the duration of uninterrupted NREM sleep time; increase the intensity of NREM sleep time; increase the duration and intensity of NREM sleep time; and treat NREM sleep disorders; by administering BYETTA® (exenatide; Amylin Pharmaceuticals, Inc., San Diego, Calif., and Eli Lilly and Co., Indianapolis, Ind.) to the patient. BYETTA® is a pharmaceutical composition that generally comprises a GLP-1 receptor agonist compound (e.g., exenatide), a preservative (e.g., metacresol), a tonicity-adjusting agent (e.g., mannitol), and a buffer (e.g., an acetate buffer).

Provided herein are methods to enhance sleep (e.g., NREM sleep); increase the duration of NREM sleep time; increase the duration of uninterrupted NREM sleep time; increase the intensity of NREM sleep time; increase the duration and intensity of NREM sleep time; and treat NREM sleep disorders; by administering exenatide once weekly (BYDUREON™, Amylin Pharmaceuticals, Inc., Eli Lilly and Company, Alkermes, Inc.) to the patient. Exenatide once weekly is a pharmaceutical composition that comprises biodegradable microspheres (e.g., poly(lactide-co-glycolide) microspheres) and exenatide. Exenatide once weekly is described, for example, in WO 2007/024700, the disclosure of which is incorporated by reference herein.

For the methods described herein, the patient may be a mammal, such as a human. In one embodiment, the human is an adult (e.g., 18 years of age or more). In one embodiment, the human is a child (e.g., less than 18 years of age). The methods described herein are useful for all patients, regardless of age. Some conditions, such as enuresis and sleep terror disorder, are usually more common in children, such that the patient in these methods of treatment is more likely to be a human child.

### BRIEF DESCRIPTION OF THE FIGURES

FIG. 1 shows the effect of exenatide on NREM sleep time. NREM sleep time (minutes±sem) was summed over 6 hour intervals following the injection of vehicle and exenatide (5, 20, 40 μg/kg). During the first 6 hours following exenatide administration, the higher doses (20 μg/kg and 40 μg/kg) elicited a clear increase in NREM sleep time compared to the vehicle condition. In hours 7-12, the action of exenatide had dissipated and NREM sleep time returned to control (i.e., vehicle) levels. Importantly, there was no residual effect of exenatide, such as a decrease in NREM sleep time (i.e., rebound insomnia) during hours 7-12. Results from repeated measured ANOVA and paired sample t-tests (**p<0.001; *p<0.01). In FIG. 1 for Hrs 1-6 and Hrs 7-12, vehicle is the bar on the far left of center; 5 μg/kg is the bar just left of center; 20 μg/kg is the bar just right of center; and 40 μg/kg is the bar on the far right of center.

FIG. 2 shows the effects of exenatide on NREM sleep time. The amount of NREM sleep time (minutes±sem) following vehicle and exenatide (20 μg/kg) administration was summed over 1 hour intervals for the 6 hour post-injection period. During each hour, vehicle was compared to each dose of exenatide using paired sample t-tests (*p<0.01). These results depict the ability of exenatide to virtually double NREM sleep time in each of the first 6 hours following administration.

FIGS. 3A and 3B show the effects of exenatide on NREM sleep time. The amount of NREM sleep time following vehicle and exenatide (5, 20, 40 μg/kg) administration was divided into 1 hour intervals for hours 1-6 (FIG. 3A) and hours 7-12 (FIG. 3B) after injection. Therefore, hour 1 (FIG. 3A) represents the first hour of the dark period. During each hour, vehicle was compared to each dose of exenatide using repeated measures ANOVA and paired sample t-tests (*p<0.01; *p<0.05). These results depict the ability of exenatide (20 μg/kg and 40 μg/kg) to increase NREM sleep time in the first 6 hours following administration. For each hour in FIGS. 3A and 3B, vehicle is the bar on the far left of center; 5 μg/kg is the bar just left of center; 20 μg/kg is the bar just right of center; and 40 μg/kg is the bar on the far right of center.

FIG. 4 provides a comparison of the NREM sleep-enhancing effect of exenatide compared to other known sleep-promoting compounds including zolpidem (e.g., AMBIEN® by Sanofi-Aventis); gaboxadol; pregabalin (e.g., LYRICA® by Pfizer); and promethazine (e.g., PENTAZINE® by Century Pharmaceuticals). In each of these studies, mice or rats were injected with the respective compound at the onset of the dark phase. The values represent the percentage increase in NREM sleep time over corresponding vehicle levels.

### DETAILED DESCRIPTION

The description provides methods of using GLP-1 receptor agonist compounds to (i) increase the duration of NREM sleep time; (ii) increase the duration of uninterrupted NREM sleep time; (iii) enhance sleep; (iv) increase the intensity of NREM sleep time; (v) increase the duration and intensity of NREM sleep time; (vi) enhance NREM sleep; (vii) treat NREM sleep disorders; or (vix) treat circadian rhythm sleep disorders in patients in need thereof. The GLP-1 receptor agonist compounds are administered to patients in therapeutically effective amounts. The patient may be a mammal, such as a human.

The description provides methods of using pharmaceutical compositions comprising GLP-1 receptor agonist compounds to (i) increase the duration of NREM sleep time; (ii) increase the duration of uninterrupted NREM sleep time; (iii) enhance sleep; (iv) increase the intensity of NREM sleep time; (v) increase the duration and intensity of NREM sleep time; (vi) enhance NREM sleep; (vii) treat NREM sleep disorders; or (vix) treat circadian rhythm sleep disorders in patients in need thereof. The pharmaceutical compositions comprise therapeutically effective amounts of the GLP-1 receptor agonist compounds. The compositions may be immediate release (e.g., administered qid, bid, tid) or extended release (e.g., administered QW or once a month). The patient may be a mammal, such as a human.

Non-rapid eye movement (NREM) sleep is dreamless sleep that makes up about 80% of sleep time. During NREM sleep, brain waves are slow, the heart rate and breathing are slow and regular, and blood pressure is low. Rapid eye movement (REM) sleep is when dreams occur and makes up about 20% of sleep time. A person experiences alternating periods of NREM and REM sleep throughout the night.

In the methods described herein, NREM sleep can be any stage, such as stage 1, stage 2, stage 3, or a combination of two or more thereof. In one embodiment, the NREM sleep is stage 1. In one embodiment, the NREM sleep is stage 2. In one embodiment, the NREM sleep is slow-wave sleep (SWS), which refers to stage 3. As of 2008, the American Academy of Sleep Medicine (AASM) discontinued the use of stage 4.

In one embodiment, the description provides methods to increase the duration of NREM sleep time by administering therapeutically effective amounts of GLP-1 receptor agonist compounds to patients in need thereof. Increasing the duration of NREM sleep time refers to a patient experiencing one or more cycles of NREM sleep for a longer period of time during the entire sleep time, when compared to the length of NREM sleep time a patient experiences without the administration of GLP-1 receptor agonist compound. In one embodiment, the method further comprises extending the duration of interrupted NREM sleep time. This refers to increasing the length of time a patient experiences NREM sleep and remains in NREM sleep without interruption.

In one embodiment, the description provides methods to increase the intensity of NREM sleep time by administering therapeutically effective amounts of GLP-1 receptor agonist compounds to patients in need thereof. Increasing the intensity of NREM sleep time also refers to increasing slow-wave sleep activity of the patient during one or more cycles of NREM sleep, when compared to the slow-wave sleep activity of a patient not being administered the GLP-1 receptor agonist compound. In one embodiment, the method further comprises increasing the intensity of interrupted NREM sleep time. This refers to increasing the length of time a patient experiences intense NREM sleep, as measured by slow-wave sleep activity. Slow-wave sleep is measured by electroencephalograph (EEG).

In one embodiment, the description provides methods to treat NREM sleep disorders by administering therapeutically effective amounts of GLP-1 receptor agonist compounds to patients in need thereof. NREM sleep disorders are those sleep disorders that occur during NREM sleep. Such disorders may also be referred to as parasomnias, and include sleepwalking disorder, sleep terror disorder, enuresis, sleep bruxism, restless leg syndrome, and periodic limb movement disorder.

In one embodiment, the NREM sleep disorder is sleep terror disorder. Sleep terror disorder is a slow-wave sleep disorder, where the patient often wakes from sleep crying or screaming. The description provides methods to treat sleep terror disorders by administering therapeutically effective amounts of GLP-1 receptor agonist compounds to patients in need of treatment for sleep terror disorders.

In one embodiment, the NREM sleep disorder is sleepwalking disorder. Sleep walking disorder is a slow-wave sleep disorder, and can be marked by sitting up in bed, walking, driving, eating, talking, or a combination of two or more thereof. The description provides methods to treat sleepwalking disorders by administering therapeutically effective amounts of GLP-1 receptor agonist compounds to patients in need of treatment for sleepwalking disorders.

In one embodiment, the NREM sleep disorder is enuresis. Enuresis is a slow-wave sleep disorder characterized by a patient urinating while sleeping. Enuresis generally occurs when a child is past the stage of potting training and generally ends in puberty. Enuresis may also be referred to as bedwetting. The description provides methods to treat enuresis by administering therapeutically effective amounts of GLP-1 receptor agonist compounds to patients in need of treatment for enuresis.

In one embodiment, the NREM sleep disorder is sleep bruxism. Sleep bruxism generally occurs during stages 1 and/or 2 of NREM sleep. Sleep bruxism is characterized by clenching the jaw and/or teeth grinding during sleep. The description provides methods to treat sleep bruxism by administering therapeutically effective amounts of GLP-1 receptor agonist compounds to patients in need of treatment for sleep bruxism.

In one embodiment, the NREM sleep disorder is restless leg syndrome. Restless leg syndrome (RLS) is a neurosensorimotor disorder characterized by paresthesias, sleep disturbances and, in most cases, periodic limb movement disorder. The description provides methods to treat restless leg syndrome by administering therapeutically effective amounts of GLP-1 receptor agonist compounds to patients in need of treatment for restless leg syndrome.

In one embodiment, the NREM sleep disorder is periodic limb movement disorder. Symptoms of periodic limb movement disorder include repeated limb movements during sleep, generally at a cycle of 5-90 second intervals, with periods of no limb movement. Many patients simultaneously experience periodic limb movement disorder and restless leg syndrome. The description provides methods to treat periodic limb movement disorder by administering therapeutically effective amounts of GLP-1 receptor agonist compounds to patients in need of treatment for periodic limb movement disorder.

In one embodiment, the description provides methods to treat circadian rhythm sleep disorders by administering therapeutically effective amounts of GLP-1 receptor agonist compounds to patients in need thereof. Exemplary circadian rhythm sleep disorders include desynchronosis (i.e., jet lag); shift work sleep disorder; delayed sleep phase disorder; advanced sleep phase syndrome; non-24-hour sleep-wake syndrome; and irregular sleep-wake pattern.

A “GLP-1 receptor agonist compound” refers to compounds having GLP-1 receptor activity. Such exemplary compounds include exendins, exendin analogs, exendin agonists, GLP-1(7-37), GLP-1(7-37) analogs, GLP-1(7-37) agonists, and the like.

The term “exendin” includes naturally occurring (or synthetic versions of naturally occurring) exendin peptides that are found in the salivary secretions of the Gila monster. Exendins of particular interest include exendin-3 and exendin-4. The exendins, exendin analogs, and exendin agonists for use in the methods described herein may optionally be amidated, and may also be in an acid form, pharmaceutically acceptable salt form, or any other physiologically active form of the molecule.

Exendin-4 (HGEGTFTSDLSKQMEEEAVRLFIEWLKNGGPSSGAPPPS—NH₂ (SEQ ID NO:1)) is a peptide found in the saliva of the Gila monster, Heloderma suspectum; and exendin-3 (HSDGTFTSDLSKQMEEEAVRLFIEWLKNGG PSSGAPPPS—NH₂ (SEQ ID NO:2)) is a peptide found in the saliva of the beaded lizard, Heloderma horridum. Exendins have some amino acid sequence similarity to some members of the glucagon-like peptide (GLP) family. For example, exendin-4 has about 53% sequence identity with glucagon-like peptide-1(GLP-1)(7-37) (HAEGTFTSDVSSYLEGQAAKEFIAWLVKGRG (SEQ ID NO:22)). However, exendin-4 is transcribed from a distinct gene, not the Gila monster homolog of the mammalian proglucagon gene from which GLP-1 is expressed. Additionally, exendin-4 is not an analog of GLP-1(7-37) because the structure of synthetic exendin-4 peptide was not created by sequential modification of the structure of GLP-1. Nielsen et al, Current Opinion in Investigational Drugs, 4(4):401-405 (2003).

Synthetic exendin-4, also known as exenatide, is commercially available as BYETTA®(Amylin Pharmaceuticals, Inc. and Eli Lilly and Company). BYETTA® contains exenatide, a preservative (e.g., metacresol), a tonicity-adjusting agent (e.g., mannitol), and a buffer (e.g., an acetate buffer). A once weekly formulation of exenatide is currently awaiting final FDA approval and is described in WO 2005/102293, the disclosure of which is incorporated by reference herein. This once weekly formulation comprises exenatide and biodegradable polymeric (e.g., poly(lactide-co-glycolide)) microspheres, and is referred to herein as EQW (BYDUREON™ by Amylin Pharmaceuticals, Inc., Eli Lilly and Company, Alkermes, Inc.).

“Exendin analog” refers to peptides or other compounds which elicit a biological activity of an exendin reference peptide, preferably having a potency equal to or better than the exendin reference peptide (e.g., exendin-4), or within five orders of magnitude (plus or minus) of potency compared to the exendin reference peptide, when evaluated by art-known measures such as receptor binding and/or competition studies as described, e.g., by Hargrove et al, Regulatory Peptides, 141:113-119 (2007), the disclosure of which is incorporated by reference herein. Preferably, the exendin analogs will bind in such assays with an affinity of less than 1 μM, and more preferably with an affinity of less than 3 nM, or less than 1 nM. The term “exendin analog” may also be referred to as “exendin agonist”.

Exendin analogs also include the peptides described herein which have been chemically derivatized or altered, for example, peptides with non-natural amino acid residues (e.g., taurine, β-amino acid residues, γ-amino acid residues, and D-amino acid residues), C-terminal functional group modifications, such as amides, esters, and C-terminal ketone modifications and N-terminal functional group modifications, such as acylated amines, Schiff bases, or cyclization, as found, for example, in the amino acid pyroglutamic acid. Exendin analogs may also contain other chemical moieties, such as peptide mimetics.

Exemplary exendins and exendin analogs exendin-4 (SEQ ID NO:1); exendin-3 (SEQ ID NO:2); Leu¹⁴-exendin-4 (SEQ ID NO:3); Leu¹⁴,Phe²⁵-exendin-4 (SEQ ID NO:4); Leu¹⁴,Ala¹⁹,Phe²⁵-exendin-4 (SEQ ID NO:5); exendin-4(1-30) (SEQ ID NO:6); Leu¹⁴-exendin-4(1-30) (SEQ ID NO:7); Leu¹⁴,Phe²⁵-exendin-4(1-30) (SEQ ID NO:8); Leu¹⁴,Ala¹⁹,Phe²⁵-exendin-4(1-30) (SEQ ID NO:9); exendin-4(1-28) (SEQ ID NO:10); Leu¹⁴-exendin-4(1-28) (SEQ ID NO:11); Leu¹⁴,Phe²⁵-exendin-4(1-28) (SEQ ID NO:12); Leu¹⁴,Ala¹⁹,Phe²⁵-exendin-4 (1-28) (SEQ ID NO:13); Leu¹⁴,Lys^{17,20},Ala¹⁹,Glu²¹,Phe²⁵,Gln²⁸-exendin-4 (SEQ ID NO:14); Leu¹⁴,Lys^{17,20},Ala¹⁹,Glu²¹,Gln²⁸-exendin-4 (SEQ ID NO:15); octylGly¹⁴,Gln²⁸-exendin-4 (SEQ ID NO:16); Leu¹⁴,Gln²⁸,octylGly³⁴-exendin-4 (SEQ ID NO:17); Phe⁴,Leu¹⁴,Gln²⁸,Lys³³,Glu³⁴, Ile^{35,36}Ser³⁷-exendin-4(1-37) (SEQ ID NO:18); Phe⁴,Leu¹⁴,Lys^{17,20},Ala¹⁹,Glu²¹,Gln²⁸-exendin-4 (SEQ ID NO:19); Val¹¹,Ile¹³,Leu¹⁴,Ala¹⁶,Lys²¹,Phe²⁵-exendin-4 (SEQ ID NO:20); exendin-4-Lys⁴⁰ (SEQ ID NO:21); lixisenatide (Sanofi-Aventis/Zealand Pharma); CJC-1134 (ConjuChem, Inc.); [N^{ε}-(17-carboxyheptadecanoic acid)Lys²⁰]exendin-4-NH₂; [N^{ε}-(17-carboxyheptadecanoyl)Lys³²]exendin-4-NH₂; [desamino-His¹,N^{ε}-(17-carboxyheptadecanoyl)Lys²⁰]exendin-4-NH₂; [Arg^{12,27},NLe¹⁴,N^{ε}-(17-carboxy-heptadecanoyl)Lys³²]exendin-4-NH₂; [N^{ε}-(19-carboxynonadecanoylamino)Lys²⁰]-exendin-4-NH₂; [N^{ε}-(15-carboxypentadecanoylamino)Lys²⁰]-exendin-4-NH₂; [N^{ε}-(13-carboxytridecanoylamino)Lys²⁰]exendin-4-NH₂; [N^{ε}-(11-carboxyundecanoyl-amino)Lys²⁰]exendin-4-NH₂; exendin-4-Lys⁴⁰(ε-MPA)-NH₂; exendin-4-Lys⁴⁰(ε-AEEA-AEEA-MPA)-NH₂; exendin-4-Lys⁴⁰(ε-AEEA-MPA)-NH₂; exendin-4-Lys⁴⁰(ε-MPA)-albumin; exendin-4-Lys⁴⁰(ε-AEEA-AEEA-MPA)-albumin; exendin-4-Lys⁴⁰(g-AEEA-MPA)-albumin; and the like. AEEA refers to [2-(2-amino)ethoxy)]ethoxy acetic acid. EDA refers to ethylenediamine. MPA refers to maleimidopropionic acid. The exendins and exendin analogs may optionally be amidated.

Other exendins and exendin analogs useful in the methods described herein include those described in WO 98/05351; WO 99/07404; WO 99/25727; WO 99/25728; WO 99/40788; WO 00/41546; WO 00/41548; WO 00/73331; WO 01/51078; WO 03/099314; U.S. Pat. No. 6,956,026; U.S. Pat. No. 6,506,724; U.S. Pat. No. 6,703,359; U.S. Pat. No. 6,858,576; U.S. Pat. No. 6,872,700; U.S. Pat. No. 6,902,744; U.S. Pat. No. 7,157,555; U.S. Pat. No. 7,223,725; U.S. Pat. No. 7,220,721; US Publication No. 2003/0036504; and US Publication No. 2006/0094652, the disclosures of which are incorporated by reference herein in their entirety.

“GLP-1(7-37) analogs” refers to peptides or other compounds which elicit a biological activity similar to that of GLP-1(7-37), when evaluated by art-known measures such as receptor binding assays or in vivo blood glucose assays as described, e.g., by Hargrove et al, Regulatory Peptides, 141:113-119 (2007), the disclosure of which is incorporated by reference herein. In one embodiment, the term “GLP-1(7-37) analog” refers to a peptide that has an amino acid sequence with 1, 2, 3, 4, 5, 6, 7 or 8 amino acid substitutions, insertions, deletions, or a combination of two or more thereof, when compared to the amino acid sequence of GLP-1(7-37). In one embodiment, the GLP-1(7-37) analog is GLP-1(7-36)-NH₂. GLP-1(7-37) analogs include the amidated forms, the acid form, the pharmaceutically acceptable salt form, and any other physiologically active form of the molecule.

Exemplary GLP-1(7-37) and GLP-1(7-37) analogs include GLP-1(7-37) (SEQ ID NO:22); GLP-1(7-36))-NH₂ (SEQ ID NO:23); liraglutide (VICTOZA® from Novo Nordisk); albiglutide (SYNCRIA® from GlaxoSmithKline); taspoglutide (Hoffman La-Roche); LY2189265 (Eli Lilly and Company); LY2428757 (Eli Lilly and Company); desamino-His⁷,Arg²⁶,Lys³⁴(N^{ε}-(γ-Glu(N-α-hexadecanoyl)))-GLP-1(7-37); desamino-His⁷,Arg²⁶,Lys³⁴(N^{ε}-octanoyl)-GLP-1(7-37); Arg^{26,34},Lys³⁸(N^{ε}(ω-carboxypentadecanoyl))-GLP-1(7-38); Arg^{26,34},Lys³⁶(N^{ε}-(γ-Glu(N-α-hexadecanoyl)))-GLP-1(7-36): Aib^{8,35},Arg^{26,34},Phe³¹-GLP-1(7-36)) (SEQ ID NO:24); HXaa₈EGTFTSDVSSYLEXaa₂₂Xaa₂₃AAKEFIXaa₃₀WLXaa₃₃Xaa₃₄G Xaa₃₆Xaa₃₇; wherein Xaa₈ is A, V, or G; Xaa₂₂ is G, K, or E; Xaa₂₃ is Q or K; Xaa₃₀ is A or E; Xaa₃₃ is V or K; Xaa₃₄ is K, N, or R; Xaa₃₆ is R or G; and Xaa₃₇ is G, H, P, or absent (SEQ ID NO:25); Arg³⁴-GLP-1(7-37) (SEQ ID NO:26); Glu³⁰-GLP-1(7-37) (SEQ ID NO:27); Lys²²-GLP-1(7-37) (SEQ ID NO:28); Gly^{8,36},Glu²²-GLP-1(7-37) (SEQ ID NO:29); Val⁸,Glu²²,Gly³⁶-GLP-1(7-37) (SEQ ID NO:30); Gly^{8,36},Glu²²,Lys³³,Asn³⁴-GLP-1(7-37) (SEQ ID NO:31); Val⁸,Glu²²,Lys³³,Asn³⁴,Gly³⁶-GLP-1(7-37) (SEQ ID NO:32); Gly^{8,36},Glu²²,Pro³⁷-GLP-1(7-37) (SEQ ID NO:33); Val⁸,Glu²²,Gly³⁶Pro³⁷-GLP-1(7-37) (SEQ ID NO:34); Gly^{8,36},Glu²²,Lys³³, Asn³⁴,Pro³⁷-GLP-1(7-37) (SEQ ID NO:35); Val⁸,Glu²²,Lys³³,Asn³⁴,Gly³⁶,Pro³⁷-GLP-1(7-37) (SEQ ID NO:36); Gly^{8,36},Glu²²-GLP-1(7-36) (SEQ ID NO:37); Val⁸,Ght²²,Gly³⁶-GLP-1(7-36) (SEQ ID NO:38); Val⁸,Glu²²,Asn³⁴,Gly³⁶-GLP-1(7-36) (SEQ ID NO:39); Gly^{8,36},Glu²²,Asn³⁴-GLP-1(7-36) (SEQ ID NO:40). Each of the GLP-1(7-37) and GLP-1(7-37) analogs may optionally be amidated.

In one embodiment, the GLP-1(7-37) or GLP-1(7-37) analogs are covalently linked (directly or by a linking group) to an Fc portion of an immunoglobulin (e.g., IgG, IgE, IgG, and the like). For example, any one of SEQ ID NOs:25-40 may be covalently linked to the Fc portion of an immunoglobulin comprising the sequence of: AESKYGPPCPPCPAPXaa₁₆Xaa₁₇Xaa₁₈GG PSVFLFPPKPKDTLMISRTPEVTCVVVDVSQEDPEVQPNWYVDGVEVH NAKTKPREEQFXaa₈₀STYRVVSVLTVLHQDWLNGKEYKCKVSNKGLPSSIEKTISKAKG QPREPQVYTLPPSQEEMTKNQVSLTCLVKGFYPSDIAVEWESNGQPENNYKTTPPVLD SDGSFFLYSRLTVDKSRWQEGNVFSCSVMHEALHNHYTQKSLSLSLGXaa₂₃₀; wherein Xaa₁₆ is P or E; Xaa₁₇ is F, V or A; Xaa₁₈ is L, E or A; Xaa₈₀ is N or A; and Xaa₂₃₀ is K or absent (SEQ ID NO:41). The linking group may be any chemical moiety (e.g., amino acids and/or chemical groups). In one embodiment, the linking group is (-GGGGS—)ₓ (SEQ ID NO:42) where x is 1, 2, 3, 4, 5 or 6; preferably 2, 3 or 4; more preferably 3. In one embodiment, the GLP-1(7-37) analog covalently linked to the Fc portion of an immunoglobulin comprises the amino acid sequence: HGEGTFTSDVSSYLEEQAAKEFIAWLVKGGGGGGGSGGGGSGGGGSA ESKYGPPCPPCPAPEAAGGPSVFLFPPKPKDTLMISRTPEVTCVVVDVSQEDPEVQ FNWYVDGVEVHNAKTKPREEQFNSTYRVVSVLTVLHQDWLNGKEYKCKVSN KGLPSSIEKTISKAKGQPREPQVYTLPPSQEEMTKNQVSLTCLVKGFYPSDIAVEWESN GQPENNYKTTPPVLDSDGSFFLYSRLTVDKSRWQEGNVFSCSVMHEALHNHYTQ KSLSLSLG (SEQ ID NO:43).

In another embodiment, the GLP-1(7-37) or GLP-1(7-37) analog may be covalently linked (directly or through a linking group) to one or two polyethylene glycol molecules. For example, a GLP-1(7-37) analog may comprise the amino acid sequence: HXaa₈EGTFTSDVS SYLEXaa₇₂QAAKEFIAWL,Xaa₃₃KGGPSSGAPPPC₄₅C₄₆-Z, wherein Xaa₈ is: D-Ala, G, V, L, I, S or T; Xaa₂₂ is G, E, I) or K; Xaa₃₃ is: V or 1; and Z is OH or (SEQ ID NO:44), and, optionally, wherein (i) one polyethylene glycol moiety is covalently attached to C₄₅, (ii) one polyethylene glycol moiety is covalently attached to C₄₆, or (iii) one polyethylene glycol moiety is attached to C₄₅ and one polyethylene glycol moiety is attached to C₄₆. In one embodiment, the GLP-1(7-37) analog is HVEGTFTSDVSSYLEEQAAKEFIAWLIKGGPSSGAPPPC₄₅C₄₆-NH₂ (SEQ ID NO:45) and, optionally, wherein (i) one polyethylene glycol moiety is covalently attached to C₄₅, (ii) one polyethylene glycol moiety is covalently attached to C₄₆, or (iii) one polyethylene glycol moiety is attached to C₄₅ and one polyethylene glycol moiety is attached to C₄₆.

In one embodiment of the methods described herein, the GLP-1 receptor agonist compounds are in a single pharmaceutical composition with another therapeutic agent. Such therapeutic agents include small molecules (e.g., sleeping aids such as zolpidem, eszopiclone, ramelteon, triazolam, zaleplon, and the like) and peptides such as amylin, amylin analogs, PYY, PYY analogs, GIP, GIP analogs, leptin, leptin analogs, and the like. In another embodiment of the methods described herein, the GLP-1 receptor agonist compounds are administered (e.g., consecutively, simultaneously, concurrently, at prescribed dosing intervals for each particular compound) in conjunction with another therapeutic agent, such as small molecules (e.g., sleeping aids such as zolpidem, eszopiclone, ramelteon, triazolam, zaleplon, and the like) and peptides such as amylin, amylin analogs, PYY, PYY analogs, GIP, GIP analogs, leptin, leptin analogs, and the like.

GLP-1 receptor agonist compounds may be prepared by processes well known in the art, e.g., peptide purification as described in Eng et al, J. Biol. Chem., 265:20259-62 (1990); standard solid-phase peptide synthesis techniques as described in Raufman et al, J. Biol. Chem., 267:21432-37 (1992); recombinant DNA techniques as described in Sambrook et al, Molecular Cloning: A Laboratory Manual, 2d Ed., Cold Spring Harbor (1989); and the like.

The disclosure also provides pharmaceutical compositions comprising the GLP-1 receptor agonist compounds described herein and a pharmaceutically acceptable carrier. The GLP-1 receptor agonist compounds can be present in the pharmaceutical composition in a therapeutically effective amount and can be present in an amount to provide a minimum blood plasma level of the GLP-1 receptor agonist compound necessary for therapeutic efficacy. Such pharmaceutical compositions are known in the art and described, e.g., in U.S. Pat. No. 7,521,423; U.S. Pat. No. 7,456,254; WO 2000/037098; WO 2005/021022; WO 2005/102293; WO 2006/068910; WO 2006/125763; WO 2009/068910; US Publication No 2004/0106547; and the like, the disclosures of which are incorporated herein by reference.

Pharmaceutical compositions containing the GLP-1 receptor agonist compounds described herein may be provided for peripheral administration, such as parenteral (e.g., subcutaneous, intravenous, intramuscular), a continuous infusion (e.g., intravenous drip, intravenous bolus, intravenous infusion), topical, nasal, or oral administration. Suitable pharmaceutically acceptable carriers and their formulation are described in standard formulation treatises, such as Remington's Pharmaceutical Sciences by Martin; and Wang et al, Journal of Parenteral Science and Technology, Technical Report No. 10, Supp. 42:2 S (1988).

The GLP-1 receptor agonist compounds described herein can be provided in parenteral compositions for injection or infusion. They can, for example, be suspended in water; an inert oil, such as a vegetable oil (e.g., sesame, peanut, olive oil, and the like); or other pharmaceutically acceptable carrier. In one embodiment, the compounds are suspended in an aqueous carrier, for example, in an isotonic buffer solution at a pH of about 3.0 to 8.0, or about 3.0 to 5.0. The compositions may be sterilized by conventional sterilization techniques or may be sterile filtered. The compositions may contain pharmaceutically acceptable auxiliary substances as required to approximate physiological conditions, such as pH buffering agents. Useful buffers include for example, acetic acid buffers. A form of repository or “depot” slow release preparation may be used so that therapeutically effective amounts of the preparation are delivered into the bloodstream over many hours or days following subcutaneous injection, transdermal injection or other delivery method. The desired isotonicity may be accomplished using sodium chloride or other pharmaceutically acceptable agents such as dextrose, boric acid, sodium tartrate, propylene glycol, polyols (such as mannitol and sorbitol), or other inorganic or organic solutes. In one embodiment for intravenous infusion, the formulation may comprise (i) the GLP-1 receptor agonist compound, (2) sterile water, and, optionally (3) sodium chloride, dextrose, or a combination thereof.

Carriers or excipients can also be used to facilitate administration of the GLP-1 receptor agonist compounds. Examples of carriers and excipients include calcium carbonate, calcium phosphate, various sugars such as lactose, glucose, or sucrose, or types of starch, cellulose derivatives, gelatin, vegetable oils, polyethylene glycols and physiologically compatible solvents.

The GLP-1 receptor agonist compounds can also be formulated as pharmaceutically acceptable salts (e.g., acid addition salts) and/or complexes thereof. Pharmaceutically acceptable salts are non-toxic salts at the concentration at which they are administered. Pharmaceutically acceptable salts include acid addition salts such as those containing sulfate, hydrochloride, phosphate, sulfamate, acetate, citrate, lactate, tartrate, methanesulfonate, ethanesulfonate, benzenesulfonate, p-toluenesulfonate, cyclohexylsulfamate and quinate. Pharmaceutically acceptable salts can be obtained from acids such as hydrochloric acid, sulfuric acid, phosphoric acid, sulfamic acid, acetic acid, citric acid, lactic acid, tartaric acid, malonic acid, methanesulfonic acid, ethanesulfonic acid, benzenesulfonic acid, p-toluenesulfonic acid, cyclohexylsulfamic acid, and quinic acid. Such salts may be prepared by, for example, reacting the free acid or base forms of the product with one or more equivalents of the appropriate base or acid in a solvent or medium in which the salt is insoluble, or in a solvent such as water which is then removed in vacuo or by freeze-drying or by exchanging the ions of an existing salt for another ion on a suitable ion exchange resin.

Exemplary pharmaceutical formulations of GLP-1 receptor agonist compounds are described in U.S. Pat. No. 7,521,423, U.S. Pat. No. 7,456,254; US Publication No 2004/0106547, WO 2006/068910, WO 2006/125763, and the like, the disclosures of which are incorporated by reference herein.

The therapeutically effective amount of the GLP-1 receptor agonist compounds described herein for use in the methods described herein will typically be from about 0.01 μg to about 5 mg; about 0.1 μg to about 2.5 mg; about 1 μg to about 1 mg; about 1 μg to about 50 μg; or about 1 μg to about 25 μg. Alternatively, the therapeutically effective amount of the GLP-1 receptor agonist compounds may be from about 0.001 μg to about 100 μg based on the weight of a 70 kg patient; or from about 0.01 μg to about 50 μg based on the weight of a 70 kg patient. These therapeutically effective doses may be administered once/day, twice/day, thrice/day, once/week, biweekly, or once/month, depending on the formulation. The exact dose to be administered is determined, for example, by the formulation, such as an immediate release formulation or an extended release formulation. For transdermal, nasal or oral dosage forms, the dosage may be increased from about 5-fold to about 10-fold.

In other embodiments of the methods described herein, the GLP-1 receptor agonist compound can be administered with (e.g., as separate compositions or in the same composition) an effective amount of an amylin, an amylin analog (e.g., davalintide), GIP, a GIP analog, PYY, a PYY analog, leptin, a leptin analog (e.g., metreleptin), or a combination of two or more thereof. Therapeutically effective amounts of these compounds are known in the art or can be determined by the skilled artisan based on the knowledge in the art and the teachings herein.

### EXAMPLES

The examples are for purposes of illustration only and are not intended to limit the scope of the disclosure or claims.

### Example 1

Male C57BL/6J mice (N=6, 4-5 months of age) were used in this experiment. The mice were surgically implanted with electrodes for EEG and electromyograph (EMG) recording of quantitative sleep-wake patterns. Mice were housed in custom-designed sleep recording chambers to control for light (12:12, L:D), temperature (24-25° C.) and noise. Food and water were available ad libitum throughout the experiment. In order to adapt mice to the injection procedure (described below) intraperitoneal (IP) injections of saline were given on 3 separate days the week prior to data collection.

To examine the effects of exenatide on sleep and wakefulness, mice received IP injections of vehicle and exenatide (5 μg/kg, 20 μg/kg, and 40 μg/kg) near the onset of the dark phase. This time of day was selected for injections because it represents the major wake period for mice, since mice are nocturnal. Therefore, dark onset is an optimal time to screen compounds for sleep-promoting effects because potentially large increases in sleep time can occur and be detected. All four injections were given to each animal using a within-subjects design and each injection trial was separated by at least 24 hours. Sleep-wake (i.e., EEG/EMG) data were collected for the 24 hours following each trial injection. Once the recordings were completed, they were analyzed to determine the amount (in minutes) of wake, NREM sleep, and REM sleep that occurred for each injection trial.

Depending on the specific analyses, sleep-wake amounts were calculated in 1 hour or 6 hour intervals for the immediate 12 hour post-injection period. This allowed for an interpretation of drug effects using different resolutions of time. Statistical comparisons were conducted using repeated measures analysis of variance (ANOVA) and/or paired sample t-tests.

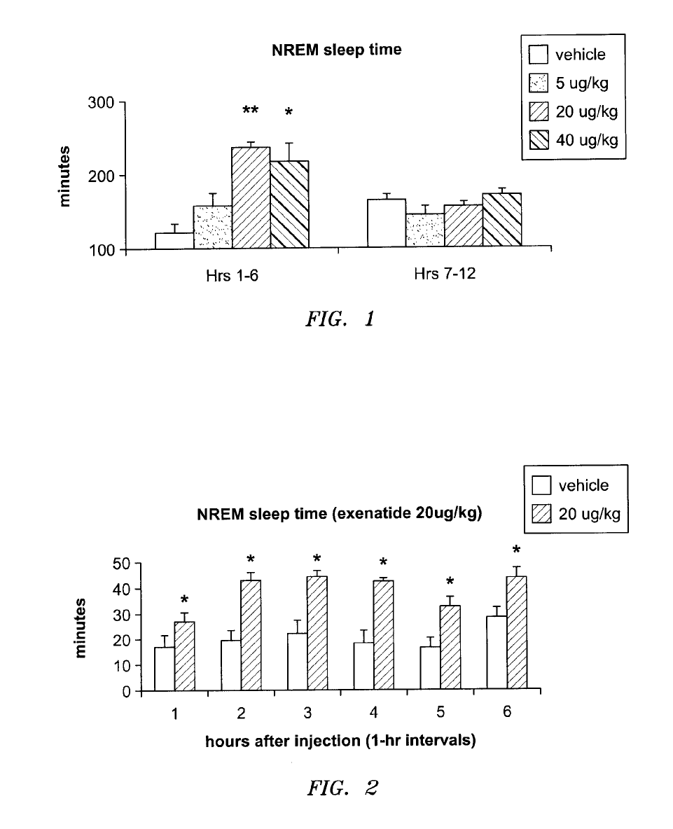

The most dramatic result of the experiment was that the amount of NREM sleep time was greatly increased following the administration of exenatide. As shown in FIG. 1, the largest increase occurred with the 20 μg/kg dose and persisted for 6 hours following the injection. During this 6 hour period, NREM sleep time almost doubled (+94%) compared to vehicle levels. A similar sleep-enhancing effect occurred at the highest dose of exenatide (40 μg/kg), which led to a 79% increase in NREM sleep time over the vehicle amount in the 6 hour post-injection period. The lowest dose of exenatide (5 μg/kg) used in this study increased NREM sleep time (+31%), but did not reach statistical significance, possibly due to the small sample size of mice.

In hours 7-12, NREM sleep time was similar between vehicle and all doses of exenatide, indicating the effect of exenatide on sleep had dissipated by this time. It is important to note that in hours 7-12, there was no indication of a negative rebound (i.e., a decrease in sleep time to below vehicle levels) in any of the exenatide conditions. This result indicates that once the sleep-promoting action of exenatide is complete, the mice return to a normal, physiological pattern of sleep.

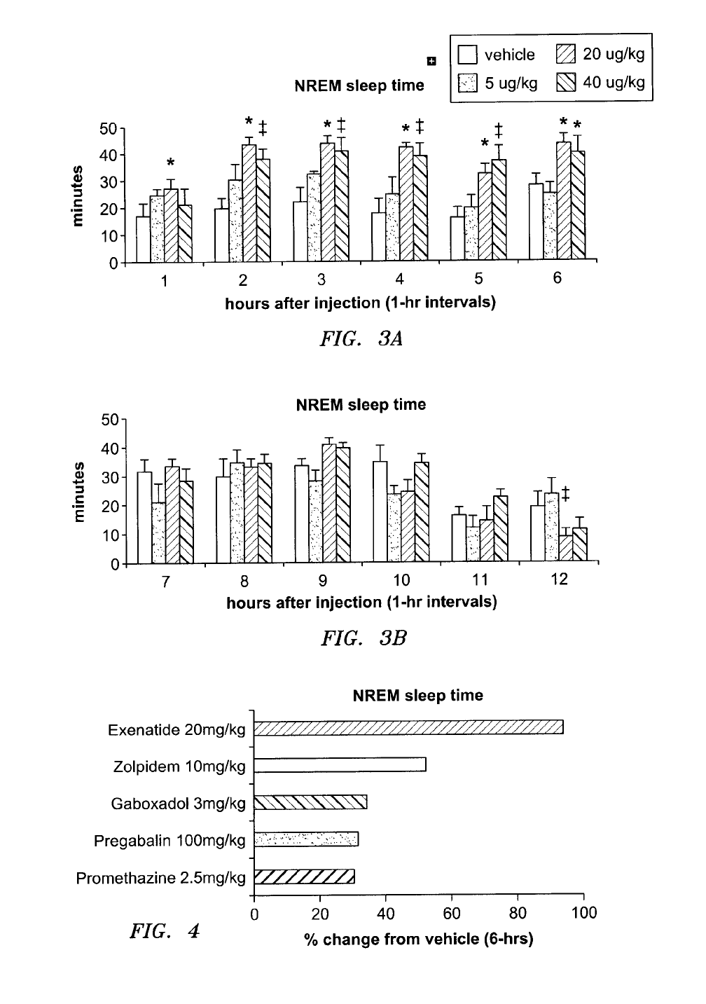

In FIG. 2, NREM sleep time is presented in 1 hour intervals for exenatide (20 μg/kg) to demonstrate the consistency of the sleep-promoting effect in each of the 6 hours following drug delivery. In addition, for a comprehensive summary of the data, FIGS. 3A and 3B show NREM sleep time following vehicle and each dose of exenatide in 1 hour intervals for the 12 hour post-injection period.

There was no significant effect of exenatide on REM sleep time (data not shown). REM sleep is very sensitive to environment as well as physiological perturbations (e.g., stress, temperature). The lack of effect of exenatide on REM sleep time served as a control indication that the mice were in a healthy physiological state following administration of the compound.

To emphasize the dramatic effect that exenatide had on sleep time in the mouse, the result of the 20 μg/kg exenatide (described above) was compared with published data (e.g., gaboxadol, pregabalin) and unpublished data (e.g., zolpidem, promethazine) of known sleep-promoting compounds on sleep in mice and rats As can be seen from FIG. 4, the percentage change over vehicle levels for the 6 hour period after treatment with exenatide is unexpectedly superior when compared to gaboxadol, pregabalin, zolpidem, and promethazine.

### Example 2

As previously demonstrated (Turek, et al., Science, 308:1043-45 (2005), there is a role of circadian behavior for internal molecular clock genes in the coordinate regulation of behavior, energy balance, and metabolism. Mice harboring a mutation in the core circadian clock gene Clock consume an increased number of small meals during their normal rest period, suggesting a defect in satiety that impacts the dysregulation of long-term energy homeostasis in these animals. The effects of exenatide, a GLP-1 receptor agonist, on circadian behavior and body weight was examined in Clock mutant mice. It was discovered that the Clock mutant mice displayed exaggerated weight loss compared to littermate control mice in response to exogenous delivery of exenatide, as well as a greater rebound weight gain following cessation of exenatide treatment. Furthermore, exenatide induced phase-shifts in the circadian rhythm of locomotor activity in wild-type animals maintained in constant darkness. Together these data show that the loss of signaling within the gut-CNS axis represents an important cause of the weight-gain and behavioral dysregulation in the Clock mutant animals.

All publications, patents, and applications are incorporated by reference herein. The foregoing has been described in detail, and the skilled artisan will recognize that modifications may be made without departing from the spirit or scope of the disclosure or appended claims.

What is claimed is:

1. A method to increase the duration of non-rapid eye movement sleep time in a patient in need thereof comprising administering to the patient a therapeutically effective amount of a GLP-1 receptor agonist compound or a pharmaceutical composition comprising a GLP-1 receptor agonist compound to increase the duration of the non-rapid eye movement sleep time.

2. A method to increase the intensity of non-rapid eye movement sleep time in a patient in need thereof comprising administering to the patient a therapeutically effective amount of a GLP-1 receptor agonist compound or a pharmaceutical composition comprising a GLP-1 receptor agonist compound to increase the intensity of the non-rapid eye movement sleep time.

3. A method to increase the duration and intensity of non-rapid eye movement sleep time in a patient in need thereof comprising administering to the patient a therapeutically effective amount of a GLP-1 receptor agonist compound or a pharmaceutical composition comprising a GLP-1 receptor agonist compound to increase the duration and intensity of the non-rapid eye movement sleep time.

4. A method to increase the duration of uninterrupted non-rapid eye movement sleep time in a patient in need thereof comprising administering to the patient a therapeutically effective amount of a GLP-1 receptor agonist compound or a pharmaceutical composition comprising a GLP-1 receptor agonist compound to increase the duration of the uninterrupted non-rapid eye movement sleep time.

5. A method to enhance sleep in a patient in need thereof comprising administering to the patient a therapeutically effective amount of a GLP-1 receptor agonist compound or a pharmaceutical composition comprising a GLP-1 receptor agonist compound to enhance sleep.

6. A method to treat a non-rapid eye movement sleep disorder in a patient in need thereof comprising administering to the patient a therapeutically effective amount of a GLP-1 receptor agonist compound or a pharmaceutical composition comprising a GLP-1 receptor agonist compound to treat the non-rapid eye movement sleep disorder.

7. The method of claim 4, wherein the non-rapid eye movement sleep disorder is sleepwalking disorder, sleep terror disorder, enuresis, sleep bruxism, restless leg syndrome, or periodic limb movement disorder.

8. A method to treat sleepwalking disorder in a patient in need thereof comprising administering to the patient a therapeutically effective amount of a GLP-1 receptor agonist compound or a pharmaceutical composition comprising a GLP-1 receptor agonist compound to treat the sleepwalking disorder.

9. A method to treat sleep terror disorder in a patient in need thereof comprising administering to the patient a therapeutically effective amount of a GLP-1 receptor agonist compound or a pharmaceutical composition comprising a GLP-1 receptor agonist compound to treat the sleep terror disorder.

10. A method to treat enuresis in a patient in need thereof comprising administering to the patient a therapeutically effective amount of a GLP-1 receptor agonist compound or a pharmaceutical composition comprising a GLP-1 receptor agonist compound to treat enuresis.

11. A method to treat sleep bruxism in a patient in need thereof comprising administering to the patient a therapeutically effective amount of a GLP-1 receptor agonist compound or a pharmaceutical composition comprising a GLP-1 receptor agonist compound to treat sleep bruxism.

12. A method to treat restless leg syndrome in a patient in need thereof comprising administering to the patient a therapeutically effective amount of a GLP-1 receptor agonist compound or a pharmaceutical composition comprising a GLP-1 receptor agonist compound to treat restless leg syndrome.

13. A method to treat periodic limb movement disorder in a patient in need thereof comprising administering to the patient a therapeutically effective amount of a GLP-1 receptor agonist compound or a pharmaceutical composition comprising a GLP-1 receptor agonist compound to treat periodic limb movement disorder.

14. The method of claim 5, wherein the sleep is non-rapid eye movement sleep.

15. The method of claim 1, 2, 4, 6, or 15, wherein the non-rapid eye movement sleep is stage 1.

16. The method of claim 1, 2, 4, 6, or 15, wherein the non-rapid eye movement sleep is stage 2.

17. The method of claim 1, 2, 4, 6, or 15, wherein the non-rapid eye movement sleep is slow-wave sleep.

18. A method to treat a circadian rhythm sleep disorder in a patient in need thereof comprising administering to the patient a therapeutically effective amount of a GLP-1 receptor agonist compound or a pharmaceutical composition comprising a GLP-1 receptor agonist compound to treat the circadian rhythm sleep disorder.

19. The method of claim 19, wherein the circadian rhythm sleep disorder is desynchronosis.

20. The method of claim 19, wherein the circadian rhythm sleep disorder is shift work sleep disorder; delayed sleep phase syndrome; advanced sleep phase syndrome; non-24-hour sleep-wake syndrome; or irregular sleep-wake pattern

21. The method of any one of claims 1-21, wherein the patient is human.

22. The method of claim 22, wherein the human is an adult.

23. The method of claim 22, wherein the human is a child.

24. The method of any one of claims 1-24, wherein the GLP-1 receptor agonist compound is an exendin, an exendin analog, GLP-1(7-37), or a GLP-1(7-37) analog.

25. The method of any one of claims 1-24, wherein the GLP-1 receptor agonist compound is exendin-4 (SEQ ID NO:1); exendin-3 (SEQ ID NO:2); Leu¹⁴-exendin-4 (SEQ ID NO:3); Leu¹⁴,Phe²⁵-exendin-4 (SEQ ID NO:4); Leu¹⁴,Ala¹⁹,Phe²⁵-exendin-4 (SEQ ID NO:5); exendin-4(1-30) (SEQ ID NO:6); Leu¹⁴-exendin-4(1-30) (SEQ ID NO:7); Leu¹⁴,Phe²⁵-exendin-4(1-30) (SEQ ID NO:8); Leu¹⁴,Ala¹⁹,Phe²⁵-exendin-4(1-30) (SEQ ID NO:9); exendin-4(1-28) (SEQ ID NO:10); Leu¹⁴-exendin-4(1-28) (SEQ ID NO:11); Leu¹⁴,Phe²⁵-exendin-4(1-28) (SEQ ID NO:12); Leu¹⁴,Ala¹⁹,Phe²⁵-exendin-4 (1-28) (SEQ ID NO:13); Leu¹⁴,Lys^{17,20},Ala¹⁹,Glu²¹Phe²⁵,Gln²⁸-exendin-4 (SEQ ID NO:14); Leu¹⁴,Lys^{17,20},Ala¹⁹,Glu²¹,Gln²⁸-exendin-4 (SEQ ID NO:15); octylGly¹⁴,Gln²⁸-exendin-4 (SEQ ID NO:16); Leu¹⁴,Gln²⁸,octylGly³⁴-exendin-4 (SEQ ID NO:17); Phe⁴,Leu¹⁴,Gln²⁸,Lys³³,Glu³⁴,Ile^{35,36},Ser³⁷-exendin-4(1-37) (SEQ ID NO:18); Phe⁴,Leu¹⁴,Lys^{17,20},Ala¹⁹,Glu²¹,Gln²⁸-exendin-4 (SEQ ID NO:19); Val¹¹,Ile¹³,Leu¹⁴,Ala¹⁶,Lys²¹,Phe²⁵-exendin-4 (SEQ ID NO:20); exendin-4-Lys⁴⁰ (SEQ ID NO:21); lixisenatide (Sanofi-Aventis/Zealand Pharma); CJC-1134 (ConjuChem, Inc.); [N^{ε}-(17-carboxyheptadecanoic acid)Lys²⁰]exendin-4-NH₂; [N^{ε}-(17-carboxyheptadecanoyl)Lys³²]exendin-4-NH₂; [desamino-His¹,N^{ε}-(17-carboxyheptadecanoyl)Lys²⁰]exendin-4-NH₂; [Arg^{12,27},NLe¹⁴,N^{ε}(17-carboxyheptadecanoyl)Lys³²]exendin-4-NH₂; [N^{ε}-(19-carboxy-nonadecanoylamino)Lys²⁰]-exendin-4-NH₂; [N^{ε}-(15-carboxypentadecanoylamino)Lys²⁰]-exendin-4-NH₂; [N^{ε}-(13-carboxytridecanoylamino)Lys²⁰]exendin-4-NH₂; [N^{ε}-(11-carboxy-undecanoylamino)Lys²⁰]exendin-4-NH₂; exendin-4-Lys⁴⁰(ε-MPA)-NH₂; exendin-4-Lys⁴⁰(ε-AEEA-AEEA-MPA)-NH₂; exendin-4-Lys⁴⁰(6-AEEA-MPA)-NH₂; exendin-4-Lys⁴⁰(6-MPA)-albumin; exendin-4-Lys⁴⁰(ε-AEEA-AEEA-MPA)-albumin; or exendin-4-Lys⁴⁰(ε-AEEA-MPA)-albumin.

26. The method of any one of claims 1-24, wherein the GLP-1 receptor agonist compound is GLP-1(7-37) (SEQ ID NO:22); GLP-1(7-36) (SEQ ID NO:23); liraglutide; albiglutide; taspoglutide; LY2189265; LY2428757; desamino-His⁷,Arg²⁶,Lys³⁴(N^{ε}-(γ-Glu(N-α-hexadecanoyl)))-GLP-1(7-37); desamino-His⁷,Arg²⁶,Lys³⁴(N^{ε}-octanoyl)-GLP-1(7-37); Arg^{26,34},Lys³⁸(N^{ε}-(ω)-carboxypentadecanoyl))-GLP-1(7-38); Arg^{26,34},Lys¹⁶(N^{ε}-(γ-Glu(N-α-hexadecanoyl)))-GLP-1(7-36); Aib^{8,35},Arg^{26,34},Phe³¹-GLP-1(7-36)) (SEQ ID NO:24); HXaa₈EGTFTSDVSSYLEXaa₂₂Xaa₂₃AAKEFIXaa₃₀WLXaa₃₃Xaa₃₄G Xaa₃₆Xaa₃₇; wherein Xaa₈ is A, V, or G; Xaa₂₂ is G, K, or E; Xaa₂₃ is Q or K; Xaa₃₀ is A or E; Xaa₃₃ is V or K; Xaa₃₄ is K, N, or R; Xaa₃₆ is R or G; and Xaa₃₇ is G, H, P, or absent (SEQ ID NO:25); Arg³⁴-GLP-1(7-37) (SEQ ID NO:26); Glu³⁰-GLP-1(7-37) (SEQ ID NO:27); Lys²²-GLP-1(7-37) (SEQ ID NO:28); Gly⁸⁻³⁶,Glu²²-GLP-1(7-37) (SEQ ID NO:29); Val⁸,Glu²²,Gly³⁶-GLP-1(7-37) (SEQ ID NO:30); Gly^{8,36},Glu²²,Lys³³,Asn³⁴-GLP-1(7-37) (SEQ ID NO:31); Val⁸,Glu²²,Lys³³,Asn³⁴,Gly³⁶-GLP-1(7-37) (SEQ ID NO:32); Gly⁸⁻³⁶,Glu²²,Pro³⁷-GLP-1(7-37) (SEQ ID NO:33); Val⁸,Glu²²,Gly³⁶Pro³⁷-GLP-1(7-37) (SEQ ID NO:34); Gly⁸³⁶,Glu²²,Lys³³, Asn³⁴,Pro³⁷-GLP-1(7-37) (SEQ ID NO:35); Val⁸,Glu²²,Lys³³,Asn³⁴,Gly³⁶ Pro³⁷-GLP-1(7-37) (SEQ ID NO:36); Gly^{8,36},Glu²²-GLP-1(7-36) (SEQ ID NO:37); Val⁸,Glu²²,Gly³⁶-GLP-1(7-36) (SEQ ID NO:38); Val⁸,Glu²²,Asn³⁴,Gly³⁶-GLP-1(7-36) (SEQ ID NO:39); or Gly^{8,36},Glu²²,Asn³⁴-GLP-1(7-36) (SEQ ID NO:40).

27. The method of any one of claims 1-24, wherein the GLP-1 receptor agonist compound is any one of SEQ ID NOs:25-40 covalently linked to the Fc portion of an immunoglobulin comprising the sequence of: AESKYGPPCPPCPAPXaa₁₆Xaa₁₇Xaa₁₈GGPSVFLFPPKPK DTLMISRTPEVTCVVVDVSQEDPEVQFNWYVDGVEVH NAKTKPREEQF Xaa₈₀STYRVVSVLTVLHQDWLNGKEYKCKVSNKGLPSSIEKTISKAKGQPREP QVYTLPPSQEEMTKNQVSLTCLVKGFYPSDIAVEWESNGQPENNYKTTPPVLD SDGSFFLYSRLTVDKSRWQEGNVFSCSVMHEALHNHYTQKSLSLSLGXaa₂₃₀; wherein Xaa₁₆ is P or E; Xaa₁₇ is F, V or A; Xaa₁₈ is L, E or A; Xaa₈₀ is N or A; and Xaa₂₃₀ is K or absent (SEQ ID NO:41).

28. The method of any one of claims 1-24, wherein the GLP-1 receptor agonist compound is HGEGTFTSDVSSYLEEQAAKEFIAWLVKGGGGGGGSGGGGSGGGGSAESKYGP PCPPCPAPEAAGGPSVFLFPPKPKDTLMISRTPEVTCVVVDVSQEDPEVQ FNWY VDGVEVHNAKTKPREEQFNSTYRVVSVLTVLHQDWLNGKEYKCKVSNKGL PSSIEKTISKAKGQPREPQVYTLPPSQEEMTKNQVSLTCLVKGFYPSDIAVEWESN GQPENNYKTTPPVLDSDGSFFLYSRLTVDKSRWQEGNVFSCSVMHEALHNHYT QKSLSLSLG (SEQ ID NO:43).

29. The method of any one of claims 1-24, wherein the GLP-1 receptor agonist compound is HXaa₈EGTFTSDVS SYLEXaa₂₂QAAKEFIAWLXaa₃₃KGGPSSGAPPPC₄₅C₄₆-Z, wherein Xaa₈ is: D-Ala, G, V, L, I, S or T; Xaa₂₂ is G, E, D or K; Xaa₃₃ is: V or I; and Z is OH or NH₂, (SEQ ID NO:44), and, optionally, wherein (i) one polyethylene glycol moiety is covalently attached to C₄₅, (ii) one polyethylene glycol moiety is covalently attached to C₄₆, or (iii) one polyethylene glycol moiety is attached to C₄₅ and one polyethylene glycol moiety is attached to C₄₆.

30. The method of any one of claims 1-24, wherein the GLP-1 receptor agonist compound is IIVEGTFTSDVSSYLEEQAAKEHAWLIKGGPSSGAPPPC₄₅C₄₆—NH₂ (SEQ ID NO:45) and, optionally, wherein (i) one polyethylene glycol moiety is covalently attached to C₄₅ (ii) one polyethylene glycol moiety is covalently attached to C₄₆, or (iii) one polyethylene glycol moiety is attached to C₄₅ and one polyethylene glycol moiety is attached to C₄₆.

31. The method of any one of claims 1-24, wherein the GLP-1 receptor agonist compound is exenatide.

32. The method of any one of claims 1-32, wherein the therapeutically effective amount of the GLP-1 receptor agonist compound is 0.01 μg to 5 mg.

33. The method of any one of claims 1-32, wherein the therapeutically effective amount of the GLP-1 receptor agonist compound is 0.1 μg to 2.5 mg.

34. The method of any one of claims 1-32, wherein the therapeutically effective amount of the GLP-1 receptor agonist compound is 1 μg to 1 mg.

35. The method of any one of claims 1-32, wherein the therapeutically effective amount of the GLP-1 receptor agonist compound is 1 μg to 50 μg.

36. The method of any one of claims 1-32, wherein the therapeutically effective amount of the GLP-1 receptor agonist compound is 1 μg to 25 μg.

37. The method of any one of claims 1-32, wherein the therapeutically effective amount of the GLP-1 receptor agonist compound is from 0.001 μg to 100 μg based on the weight of a 70 kg patient.

38. The method of any one of claims 1-32, wherein the therapeutically effective amount of the GLP-1 receptor agonist compound is from 0.01 μg to 50 μg based on the weight of a 70 kg patient.

39. The method of any one of claims 1-39, wherein the pharmaceutical composition comprises the GLP-1 receptor agonist compound, a preservative, a tonicity-adjusting agent and a buffer; and wherein the GLP-1 receptor agonist compound is exenatide.

40. The method of any one of claim 1-39, wherein the pharmaceutical composition comprises the GLP-1 receptor agonist compound, metacresol, mannitol, and an acetate buffer; wherein the GLP-1 receptor agonist compound is exenatide.

41. The method of any one of claims 1-39, wherein the pharmaceutical composition comprises biodegradable microspheres comprising the GLP-1 receptor agonist compound; wherein the GLP-1 receptor agonist compound is exenatide.

42. The method of claim 42, wherein the biodegradable microspheres are poly(lactide-co-glycolide) microspheres.

43. The method of any one of claims 1-43, further comprising administering an effective amount of an amylin, an amylin analog, GIP, a GIP analog, PYY, a PYY analog, leptin, a leptin analog, or a combination of two or more thereof.
